(19) United States
(12) Patent Application Publication
(10) Pub. No.: US 2004/0081967 A1
(43) Pub. Date: Apr. 29, 2004

(54) Chemical arrays with features of different probe densities

(76) Inventors: Eric M. Leproust, Campbell, CA (US); Bill J. Peck, Mountain View, CA (US)

Correspondence Address: AGILENT TECHNOLOGIES, INC., Legal Department, DL429, Intellectual Property Administration, P.O. Box 7599, Loveland, CO, 80537-0599, US

(21) Appl. No.: 10/281,408

(22) Filed: Oct. 25, 2002

(51) Int. Cl. B05D 3/00; C12Q 1/68; C12M 1/34

(52) U.S. Cl. 435/6; 435/287.2; 427/2.11

(57) ABSTRACT

A method of fabricating one or more arrays of multiple chemical probes bound to a surface of a substrate at different features of the array. The method may include contacting each of at least ten different probes or probe precursors with different locations on the substrate surface, so that each of the probes or probe precursors binds to the different locations through the linker agent, and repeating this as needed. The one or more arrays are therefore formed with at least ten features of different probe composition which are repeated with a different probe density. Arrays, other methods, apparatus and computer program products are further provided.

### FIELD OF THE INVENTION

This invention relates to arrays, such as polynucleotide arrays (for example, DNA arrays), which are useful in diagnostic, screening, gene expression analysis, and other applications.

### BACKGROUND OF THE INVENTION

In the following discussion and throughout the present application, while various references are cited no cited reference is admitted to be prior art to the present application.

Chemical arrays, such as polynucleotide or protein arrays (for example, DNA or RNA arrays), are known and are used, for example, as diagnostic or screening tools. Polynucleotide arrays include regions of usually different sequence polynucleotides arranged in a predetermined configuration on a substrate. These regions (sometimes referenced as “features”) are positioned at respective locations (“addresses”) on the substrate. The arrays, when exposed to a sample, will exhibit an observed binding pattern. This binding pattern can be detected upon reading the array. For example all polynucleotide targets (for example, DNA) in the sample can be labeled with a suitable label (such as a fluorescent compound), and the fluorescence pattern on the array accurately observed following exposure to the sample. Assuming that the different sequence polynucleotides were correctly deposited in accordance with the predetermined configuration, then the observed binding pattern will be indicative of the presence and/or concentration of one or more polynucleotide components of the sample.

Biopolymer arrays can be fabricated by depositing previously obtained biopolymers (such as from synthesis or natural sources) onto a substrate, or by in situ synthesis methods. Methods of depositing obtained biopolymers include loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. Such a deposition method can be regarded as forming each feature by one cycle of attachment (that is, there is only one cycle at each feature during which the previously obtained biopolymer is attached to the substrate). For in situ fabrication methods, multiple different reagent droplets are deposited by pulse jet or other means at a given target location in order to form the final feature (hence a probe of the feature is synthesized on the array substrate). The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. The in situ method for fabricating a polynucleotide array typically follows, at each of the multiple different addresses at which features are to be formed, the same conventional iterative sequence used in forming polynucleotides from nucleoside reagents on a support by means of known chemistry. This iterative sequence can be considered as multiple ones of the following attachment cycle at each feature to be formed: (a) coupling an activated selected nucleoside (a monomeric unit) through a phosphite linkage to a functionalized support in the first iteration, or a nucleoside bound to the substrate (i.e. the nucleoside-modified substrate) in subsequent iterations; (b) optionally, blocking unreacted hydroxyl groups on the substrate bound nucleoside (sometimes referenced as “capping”); (c) oxidizing the phosphite linkage of step (a) to form a phosphate linkage; and (d) removing the protecting group (“deprotection”) from the now substrate bound nucleoside coupled in step (a), to generate a reactive site for the next cycle of these steps. The coupling can be performed by depositing drops of an activator and phosphoramidite at the specific desired feature locations for the array. A final deprotection step is provided in which nitrogenous bases and phosphate group are simultaneously deprotected by treatment with ammonium hydroxide and/or methylamine under known conditions. Capping, oxidation and deprotection can be accomplished by treating the entire substrate (“flooding”) with a layer of the appropriate reagent. The functionalized support (in the first cycle) or deprotected coupled nucleoside (in subsequent cycles) provides a substrate bound moiety with a linking group for forming the phosphite linkage with a next nucleoside to be coupled in step (a). Final deprotection of nucleoside bases can be accomplished using alkaline conditions such as ammonium hydroxide, in another flooding procedure in a known manner. Conventionally, a single pulse jet or other dispenser is assigned to deposit a single monomeric unit.

The foregoing chemistry of the synthesis of polynucleotides is described in detail, for example, in Caruthers, Science 230: 281-285, 1985; Itakura et al., Ann. Rev. Biochem. 53: 323-356; Hunkapillar et al., Nature 310: 105-110, 1984; and in “Synthesis of Oligonucleotide Derivatives in Design and Targeted Reaction of Oligonucleotide Derivatives”, CRC Press, Boca Raton, Fla., pages 100 et seq., U.S. Pat. No. 4,458,066, U.S. Pat. No. 4,500,707, U.S. Pat. No. 5,153,319, U.S. Pat. No. 5,869,643, EP 0294196, and elsewhere. The phosphoramidite and phosphite triester approaches are most broadly used, but other approaches include the phosphodiester approach, the phosphotriester approach and the H-phosphonate approach. The substrates are typically functionalized to bond to the first deposited monomer. Suitable techniques for functionalizing substrates with such linking moieties are described, for example, in U.S. Pat. No. 6,258,454 and Southern, E. M., Maskos, U. and Elder, J. K., Genomics, 13, 1007-1017, 1992. In the case of array fabrication, different monomers and activator may be deposited at different addresses on the substrate during any one cycle so that the different features of the completed array will have different desired biopolymer sequences. One or more intermediate further steps may be required in each cycle, such as the conventional oxidation, capping and washing steps in the case of in situ fabrication of polynucleotide arrays (again, these steps may be performed in flooding procedure).

Further details of fabricating biopolymer arrays by depositing either previously obtained biopolymers or by the in situ method are disclosed in U.S. Pat. No. 6,242,266, U.S. Pat. No. 6,232,072, U.S. Pat. No. 6,180,351, and U.S. Pat. No. 6,171,797. In array fabrication, the quantities of polynucleotide available are usually very small and expensive. Additionally, sample quantities available for testing are usually also very small and it is therefore desirable to simultaneously test the same sample against a large number of different probes on an array. These conditions require use of arrays with large numbers of very small, closely spaced features.

In fabricating arrays by depositing previously obtained biopolymers or by the in situ method, typically the entire region on the substrate surface on which an array will be formed (an “array region”) is completely exposed to one or more reagents. For example, in either method array regions will often be exposed to one or more linker compositions to form a suitable linker layer on the surface which binds to both the substrate and biopolymer or biomonomer. Particularly useful linker compositions and methods are disclosed in U.S. Pat. Nos. 6,319,674 and 6,444,268 which may use various silane based compounds as linkers or other surface modifying agents (for example, to modify the surface energy to control deposited drop spread). The solution containing the silane compounds is exposed to a substrate surface in a reactor.

An array is typically read such as by detecting light emitted from features in response to an interrogating light. A typical detector may be a photomultiplier tube (“PMT”). However, the concentration of the various target polynucleotides (or other targets) in a sample is not always known in advance. On the other hand, the present invention now recognizes that each feature can only quantitatively measure a limited range of target concentrations (sometimes referenced herein as the “dynamic range” of a feature). For example, features having a higher probe density tend to be relatively insensitive to changes in target concentrations at lower levels whereas features having a lower probe density tend to be relatively insensitive to changes in target concentration at higher levels.

The present invention then, recognizes that it would be desirable to extend the dynamic range over which changes in a target concentration can be detected by a chemical array.

### SUMMARY OF THE INVENTION

The present invention then provides one or more arrays of multiple chemical probes bound to a surface of a substrate at different features of the array. The arrays may have features of different probe composition (for example, at least ten such features) which are repeated at different probe density.

In one configuration of the one or more arrays of the present invention, different regions (for example, at least two or three regions) of the surface each may each have multiple features (for example, at least ten or at least one hundred) with a same probe density within a region and the different regions have different probe densities. These different regions may also have different linker agent densities, the probes being bound to the different regions through the linker agent.

The one or more arrays of the present invention may be provided as a component of an array assembly which has an associated indication of features having different probe densities of a same probe composition. The associated indication can, for example, comprise one or more identifiers on the substrate which carry the indication or one or more identifiers on the array which is linked to a file which carries the indication. The present invention further provides a method of producing a surface modified substrate. This method includes comprising contacting different regions of the substrate surface to a linking agent under different conditions (for example, by contacting those different regions with fluids having different linking agent concentrations) so that the linking agent binds to the different regions at different densities.

There is also provided by the present invention, a method of fabricating one or more arrays of multiple chemical probes bound to a surface of a substrate at different features of the array. This method may include contacting different probes or probe precursors (for example, at least ten or one hundred of them) with different locations on the substrate surface, so that each of the probes or probe precursors binds to the different locations through the linker agent. The foregoing can be repeated as needed so as to form the one or more arrays with features of different probe composition which are repeated with a different probe density.

The present invention further provides a method which includes reading one or more chemical arrays of the present invention, each of which has been exposed to a sample, to obtain signal data from the features. The signal data from the features of the same probe composition but with different probe densities, may then be merged.

There is further provided by the present invention, apparatus, and computer program products, which can execute one or more methods of the present invention for making substrates or arrays, or for reading arrays.

The various aspects of the present invention can provide any one or more of the following and/or other useful benefits. For example, arrays can be provided which can detect a target for a particular probe over a high dynamic range.

### BRIEF DESCRIPTION OF THE DRAWINGS

FIG. 1 illustrates an array assembly carrying multiple arrays, such as may be fabricated by methods of the present invention;

FIG. 2 is an enlarged view of a portion of FIG. 1 showing multiple ideal spots or features of an array;

FIG. 3 is an enlarged illustration of a portion of FIG. 2;

FIG. 4 illustrates a method of depositing drops of a surface linking agent, additional agent such as surface energy modifier, or solvent, to produce a region of a desired linking agent and capping agent density;

FIGS. 5 and 6 show some different array assemblies of the present invention;

FIG. 7 illustrates an apparatus of the present invention which can execute a method of the present invention for producing a substrate or fabricating arrays;

FIG. 8 illustrates an array reader of the present invention;

FIG. 9 is a flowchart illustrating methods of the present invention; and

FIG. 10 illustrates a way in which arrays of the present invention may provide a high dynamic range.

To facilitate understanding, the same reference numerals have been used, where practical, to designate the same elements that are common to the figures. Different letters, after the same number indicate members of a generic class (for example, arrays 12a, 12b may be collectively referred to as “arrays 12”). Drawings are not necessarily to scale.

### DETAILED DESCRIPTION OF EMBODIMENTS OF THE INVENTION

In the present application, unless a contrary intention appears, the following terms refer to the indicated characteristics. A “biopolymer” is a polymer of one or more types of repeating units. Biopolymers are typically found in biological systems and particularly include polysaccharides (such as carbohydrates), and peptides (which term is used to include polypeptides, and proteins whether or not attached to a polysaccharide) and polynucleotides as well as their analogs such as those compounds composed of or containing amino acid analogs or non-amino acid groups, or nucleotide analogs or non-nucleotide groups. This includes polynucleotides in which the conventional backbone has been replaced with a non-naturally occurring or synthetic backbone, and nucleic acids (or synthetic or naturally occurring analogs) in which one or more of the conventional bases has been replaced with a group (natural or synthetic) capable of participating in Watson-Crick type hydrogen bonding interactions. Polynucleotides include single or multiple stranded configurations, where one or more of the strands may or may not be completely aligned with another. A “nucleotide” refers to a sub-unit of a nucleic acid and has a phosphate group, a 5 carbon sugar and a nitrogen containing base, as well as functional analogs (whether synthetic or naturally occurring) of such sub-units which in the polymer form (as a polynucleotide) can hybridize with naturally occurring polynucleotides in a sequence specific manner analogous to that of two naturally occurring polynucleotides. For example, a “biopolymer” includes DNA (including cDNA), RNA, oligonucleotides, and PNA and other polynucleotides as described in U.S. Pat. No. 5,948,902 and references cited therein (all of which are incorporated herein by reference), regardless of the source. An “oligonucleotide” generally refers to a nucleotide multimer of about 10 to 100 nucleotides in length, while a “polynucleotide” includes a nucleotide multimer having any number of nucleotides. A “biomonomer” references a single unit, which can be linked with the same or other biomonomers to form a biopolymer (for example, a single amino acid or nucleotide with two linking groups one or both of which may have removable protecting groups). A biomonomer fluid or biopolymer fluid reference a liquid containing either a biomonomer or biopolymer, respectively (typically in solution).

An “array”, unless a contrary intention appears, includes any one, two or three-dimensional arrangement of addressable regions bearing a particular chemical moiety or moieties (for example, biopolymers such as polynucleotide sequences) associated with that region. Each region may extend into a third dimension in the case where the substrate is porous while not having any substantial third dimension measurement (thickness) in the case where the substrate is non-porous. An array is “addressable” in that it has multiple regions of different moieties (for example, different polynucleotide sequences) such that a region (a “feature” or “spot” of the array) at a particular predetermined location (an “address”) on the array will detect a particular target or class of targets (although a feature may incidentally detect non-targets of that feature). An array feature is generally homogenous and the features typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “target probes” may be the one which is to be evaluated by the other (thus, either one could be an unknown mixture of polynucleotides to be evaluated by binding with the other). An “array layout” or “array characteristics”, refers to one or more physical, chemical or biological characteristics of the array, such as feature positioning, one or more feature dimensions, or some indication of an identity or function (for example, chemical or biological) of a moiety at a given location, or how the array should be handled (for example, conditions under which the array is exposed to a sample, or array reading specifications or controls following sample exposure). “Hybridizing” and “binding”, with respect to polynucleotides, are used interchangeably.

A “plastic” is any synthetic organic polymer of high molecular weight (for example at least 1,000 grams/mole, or even at least 10,000 or 100,000 grams/mole.

“Flexible” with reference to a substrate or substrate web, references that the substrate can be bent 180 degrees around a roller of less than 1.25 cm in radius. The substrate can be so bent and straightened repeatedly in either direction at least 100 times without failure (for example, cracking) or plastic deformation. This bending must be within the elastic limits of the material. The foregoing test for flexibility is performed at a temperature of 20° C.

A “web” references a long continuous piece of substrate material having a length greater than a width. For example, the web length to width ratio may be at least 5/1, 10/1, 50/1, 100/1, 200/1, or 500/1, or even at least 1000/1.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data. An array “assembly” may be the array plus only a substrate on which the array is deposited, although the assembly may be in the form of a package which includes other features (such as a housing with a chamber). A “chamber” references an enclosed volume (although a chamber may be accessible through one or more ports). It will also be appreciated that throughout the present application, that words such as “front”, “back”,“top”, “upper”, and “lower” are used in a relative sense only. “Fluid” is used herein to reference a liquid. Reference to a singular item, includes the possibility that there are plural of the same items present. “May” refers to optionally. Any recited method can be carried out in the ordered sequence of events as recited, or any other logically possible sequence.

A “pulse jet” is any device which can dispense drops in the formation of an array. Pulse jets operate by delivering a pulse of pressure (such as by a piezoelectric or thermoelectric element) to liquid adjacent an outlet or orifice such that a drop will be dispensed therefrom.

A “linking layer” bound to the surface may, for example, be less than 200 angstroms or even less than 10 angstroms in thickness (or less than 8, 6, or 4 angstroms thick). Such layer may have a polynucleotide, protein, nucleoside or amino acid minimum binding affinity of 10⁴ to 10⁶ units/μ². Layer thickness can be evaluated using UV or X-ray elipsometry.

“Continuous” in reference to an area on the substrate surface references an area which is uninterrupted by any gaps within that area. The distinct features of an array may then be formed on such a continuous area.

A “group” in relation to a chemical formula, includes both substituted and unsubstituted forms of the group.

“Lower alkyl group” is an alkyl group with from 1 to 6 C atoms, and may only have any one of 1, 2, 3, or 4 C atoms.

“Surface energy” is as defined in U.S. Pat. No. 6,444,268.

A “region” on a substrate surface is a continuous area on that surface, with different regions not overlapping one another. Typically, a particular region will contain multiple features (such as at least ten, at least fifty, at least one or two hundred, or at least one thousand) of the same probe density. Each region may have an area of at least 1 mm², or at least 10 mm², at least 100 mm², or at least 200 mm².

“Linker agent density” or “capping agent density” refers to the number of linker molecules or capping molecules per unit area. Linker agents are counted in determining linker agent density whether or not they are linked to probes or are themselves capped. For capping agent density only capping agents directly attached to the substrate surface are counted in the capping agent density. Linker agent density within a region includes linker agent within any interfeature areas, and will typically be relatively uniform over a given region, although there may be some minor variation. If different regions on a substrate surface of uniform composition are exposed under the same conditions to a same composition of linking agent which binds to the surface, the linker agent density in the regions will be considered to be the “same”. These terms are used interchangeably with, and have the same means as, “region linker agent density” and “region capping agent density”.

“Probe density” is a shorthand way of referring to the number of linker molecules or probe molecules per unit area within a feature. This term then is used interchangeably with, and has the same meaning as “feature probe density”. Thus, any interfeature areas which are essentially devoid of the probe are not taken into consideration in determining a probe density. “Probe density” in a region then, is distinct and independent of feature density (which is the number of features per unit area).

A different linker agent density, different capping agent density, or different probe density means the average linker agent, capping agent, or probe density over the area referenced differs by more than 5%, for example more than 10%, 15%, 20% or 50%. A “same” density of any of the foregoing means that the average linker agent, capping agent, or probe density over the area referenced differs by less than 20%, for example no more than 10%, 5%, 2% or 1%.

The steps of any method herein may be performed in the recited order, or in any other order that is logically possible. All patents and other references cited in this application, are incorporated into this application by reference except insofar as anything in those patents or references, including definitions, conflicts with anything in the present application (in which case the present application is to prevail).

Turning now to arrays of the present invention, these may have multiple features of higher probe density which are formed on a region of higher linking agent density and multiple features of lower probe density are formed on a region of lower linking agent density. Different regions of different linking agent density may also have a capping agent present, with a lower density of capping agent at a region which has a higher density of linker agent. The number of different regions may be at least two, at least three, at least four, at least five, or at least ten. The number of features of different probe composition which are present on each region of higher linking agent density and which features are repeated at a lower probe density on a region of lower linking agent density, may be at least five, at least ten, at least one hundred, or at least one or two thousand.

As to methods of the present invention for producing a surface modified substrate, such methods may also include contacting the different regions on the substrate surface with a capping agent. The capping agent will also bind to the different regions to produce a substrate surface having both linking agent and capping agent bound to the different regions with a lower density of capping agent at a region which has a higher density of linking agent. However, the capping agent will not bind to a subsequently deposited probe and so it “caps” or blocks any reactive sites on the substrate surface. The capping agent may be in a fluid different from that containing the linking agent which contacts the substrate surface, or may be in the same fluid as the linking agent with, for example, a fluid with a higher concentration of linking agent containing a lower concentration of capping agent. In any event, one way of contacting the fluids containing the linking agent or capping agent is by depositing drops of the fluid to be deposited at the different regions so that the drops for each region together continuously cover that region.

The probes of the arrays may be any biopolymer, such as polynucleotides or amino acid polymers (which term is used to include peptides and proteins). During array fabrication, a same concentration of a same probe or probe precursor may be used to contact the locations of repeated features of different probe density. For example, when the foregoing different locations of repeated features are in different regions with a different density of a same or different linking agent, this will result in features of a probe composition which are repeated in the different regions at different feature probe density. This is one way to provide the different regions each with the features of a same feature probe density.

Arrays may also be fabricated which, as already described, may have different regions with different densities of a same or different linker agent present (to which probes will be bound) and optionally, capping agent (to which probes will not bind). Since the different regions may have different surface energy levels and therefore different contact angles with drops of the same composition comprising the probe or probe precursors, drops of a larger volume may be deposited on one of the regions having a higher contact angle than are deposited at another one of the regions having a lower contact angle. This may help reduce disparity in feature areas in the different regions.

The apparatus of the present invention may include a substrate holder on which the substrate can be mounted, and a drop deposition system to deposit drops of the probes or probe precursors. A processor controls the drop deposition system so as to contact each of the different probes or probe precursors with different locations on the substrate surface, and repeat this as needed, so as to form the array. The processor may also receive an indication of the location of different regions on a substrate to which probes or probe precursors will bind with different density, and controls the drop deposition system to form the array with features of different probe composition in one of the regions which are repeated in another of the regions at a different probe density.

Computer program products of the present invention may include a computer readable storage medium having a computer program stored thereon which controls the apparatus to perform a method as described herein. Any computer readable storage medium for any purpose herein may include, for example, an optical or magnetic memory (such as a fixed or portable disk or other device), or a solid state memory.

### Arrays and Surface Modified Substrates

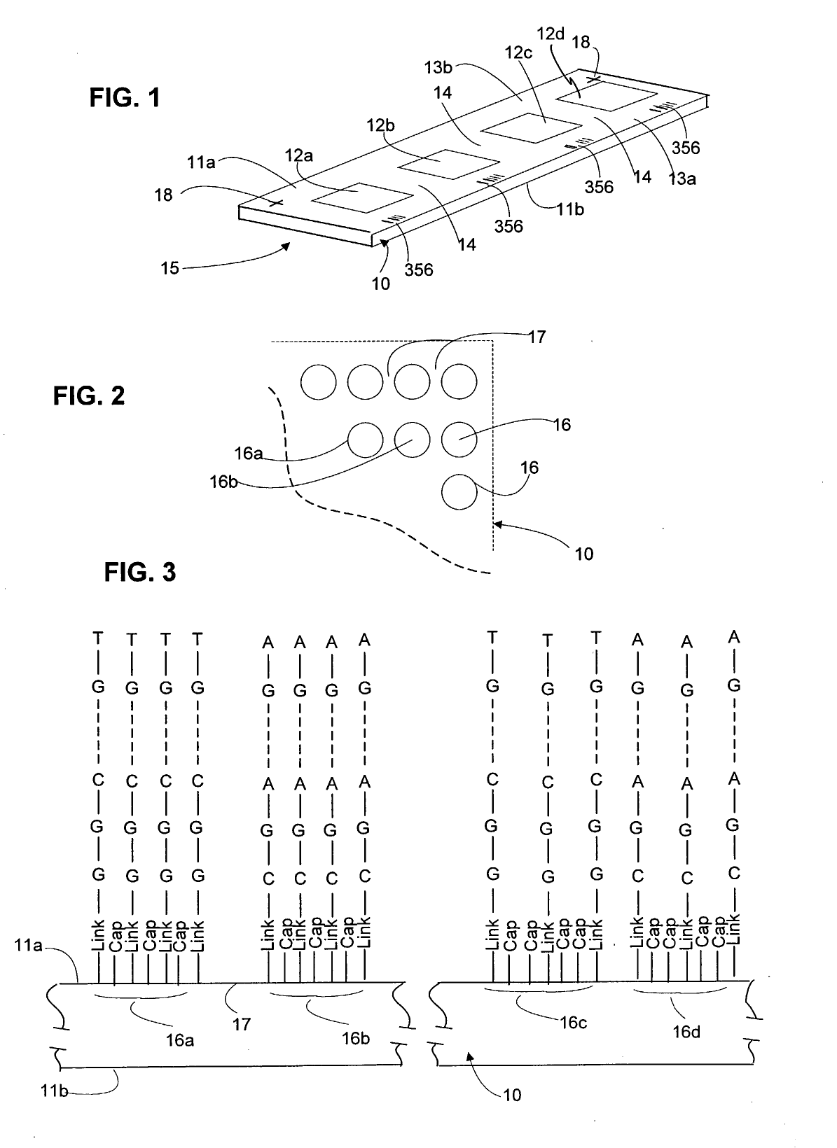

Referring first to FIGS. 1-3, an array assembly 15 of the present invention may include a substrate which can be, for example, in the form of an a rigid substrate 10 (for example, a transparent non-porous material such as glass or silica) of limited length, carrying one or more arrays 12 disposed along a front surface 11a of substrate 10 and separated by inter-array areas 14. Throughout this application any different members of a generic class may have the same reference number followed by different letters (for example, arrays 12a, 12b, 12c, and 12d may generically be referenced as “arrays 12”) Alternatively, substrate 10 can be flexible. Each array 12 occupies its own region on surface 11a which is co-extensive with the array (hence the regions do not extend into areas 14). A back side 11b of substrate 10 does not carry any arrays 12. The arrays on substrate 10 can be designed for testing against any type of sample, whether: a trial sample; reference sample;, a combination of the foregoing; or a known mixture of polynucleotides, proteins, polysaccharides and the like (in which cases the arrays may be composed of features carrying unknown sequences to be evaluated). While four arrays 12 are shown in FIG. 1, it will be understood that substrate 10 and the embodiments to be used with it, may use any number of desired arrays 12 such as at least one, two, five, ten, twenty, fifty, or one hundred (or even at least five hundred, one thousand, or at least three thousand). When more than one array 12 is present they may be arranged end to end along the lengthwise direction of substrate 10. Depending upon intended use, any or all of arrays 12 may be the same or different from one another and each will contain multiple spots or features 16 of biopolymers in the form of polynucleotides.

A typical array 12 may contain from more than ten, more than one hundred, more than one thousand or ten thousand features, or even more than from one hundred thousand features. For example, features may have widths (that is, diameter, for a round spot) in the range from a 10 μm to 1.0 cm. In other embodiments each feature may have a width in the range of 1.0 μm to 1.0 mm, usually 5.0 μm to 500 μm, and more usually 10 μm to 200 μm. Non-round features may have area ranges equivalent to that of circular features with the foregoing width (diameter) ranges. At least some, or all, of the features are of different. compositions (for example, when any repeats of each feature of the same composition are excluded, the remaining features may account for at least 5%, 10%, or 20% of the total number of features).

Each array 12 may cover an area of less than 100 cm², or even less than 50 cm², 10 cm² or 1 cm². In many embodiments, particularly when substrate 10 is rigid, it may be shaped generally as a rectangular solid (although other shapes are possible), having a length of more than 4 mm and less than 1 m, usually more than 4 mm and less than 600 mm, more usually less than 400 mm; a width of more than 4 mm and less than 1 m, usually less than 500 mm and more usually less than 400 mm; and a thickness of more than 0.01 mm and less than 5.0 mm, usually more than 0.1 mm and less than 2 mm and more usually more than 0.2 and less than 1 mm. When substrate 10 is flexible, it may be of various lengths including at least 1 m, at least 2 m, or at least 5 m (or even at least 10 m). With arrays that are read by detecting fluorescence, the substrate 10 may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, substrate 10 may transmit at least 20%, or 50% (or even at least 70%, 90%, or 95%), of the illuminating light incident on the front as may be measured across the entire integrated spectrum of such illuminating light or alternatively at 532 nm or 633 nm.

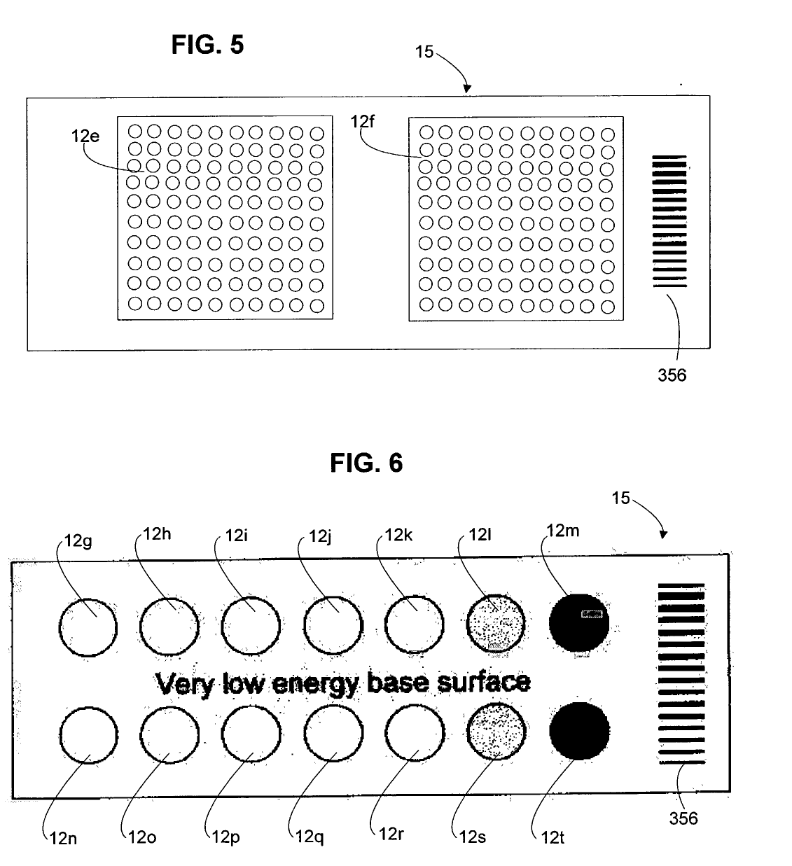

In the case where arrays 12 are formed by the conventional in situ or deposition of previously obtained moieties, as described above, by depositing for each feature a droplet of reagent in each cycle such as by using a pulse jet such as an inkjet type head, interfeature areas 17 will typically (but not essentially) be present which do not carry any polynucleotide. It will be appreciated though, that the interfeature areas 17 could be of various sizes and configurations. It will also be appreciated that there need not be any space separating arrays 12 from one another (for example, when arrays are fabricated using light directed techniques). Each feature carries a predetermined polynucleotide (which includes the possibility of mixtures of polynucleotides). As per usual, A, C, G, T represent the usual nucleotides. “Link” (see FIG. 3 in particular) represents a linking agent (molecule) covalently bound to the front surface and a first nucleotide, as provided by a method of the present invention and as further described below.

Substrate 10 also one or more identifiers in the form of bar codes 356. Identifiers such as other optical or magnetic identifiers could be used instead of bar codes 356 which will carry the information discussed below. Each identifier may be associated with its corresponding array by being positioned adjacent that array 12. However, this need not be the case and identifiers such as bar code 356 can be positioned elsewhere on substrate 10 if some other means of associating each bar code 356 with its corresponding array is provided (for example, by relative physical locations). Further, a single identifier might be provided which is associated with more than one array 12 on a same substrate 10 and such one or more identifiers may be positioned on a leading or trailing end of substrate 10. The substrate may further have one or more fiducial marks 18 for alignment purposes during array fabrication.

FIGS. 2 and 3 illustrate ideal features 16 of an array 12 where the actual features formed are the same as the target (or “aim”) features, with each feature 16 being uniform in shape, size and composition, and the features being regularly spaced. Such an array when fabricated by drop deposition methods, would require all reagent droplets for each feature to be uniform in shape and accurately deposited at the target feature location. In practice, such an ideal result may be difficult to obtain due to fixed and random errors during fabrication.

One or more of the arrays may be duplicated on surface 11a with the same features except that features of the same composition may have different feature probe density. In one example of this, in the configuration of FIG. 1 arrays 12a, 12b are different arrays with at least some (or all) features of different probe composition. Array 12c repeats some or all of the features of array 12a but at a lower feature probe density in each feature than in array 12a, while array 12d repeats some (or all) of the features of array 12b but also at a lower feature probe density than in array 12b. All features within a same array have the same feature probe density, with probes bound to surface 11a through linker agents identified as “Link” in FIG. 3. A capping agent is also present on each of the features 12a-12d. Note that in this particular example all features in arrays 12a, 12b have the same feature probe density and linker agent density while those features in arrays 12b, 12c also have the same feature probe density and linker agent density both of which are lower than in arrays 12a, 12b. Also, the different regions occupied by arrays 12c, 12d have a capping agent density which is lower than the capping agent density at the regions occupied by arrays 12a, 12b. The foregoing relative densities can be best seen in FIG. 3 wherein feature 16a is located in array 12a, feature 16b in array 12b, feature 16c in array 12c, and feature 16d in array 12d. Thus, features 16a, 16c are features of a same probe composition but with different feature probe densities and different region linker agent/capping agent densities. Similarly features 16b, 16d are of a same probe composition but with different feature probe density and different region linker agent/capping agent densities.

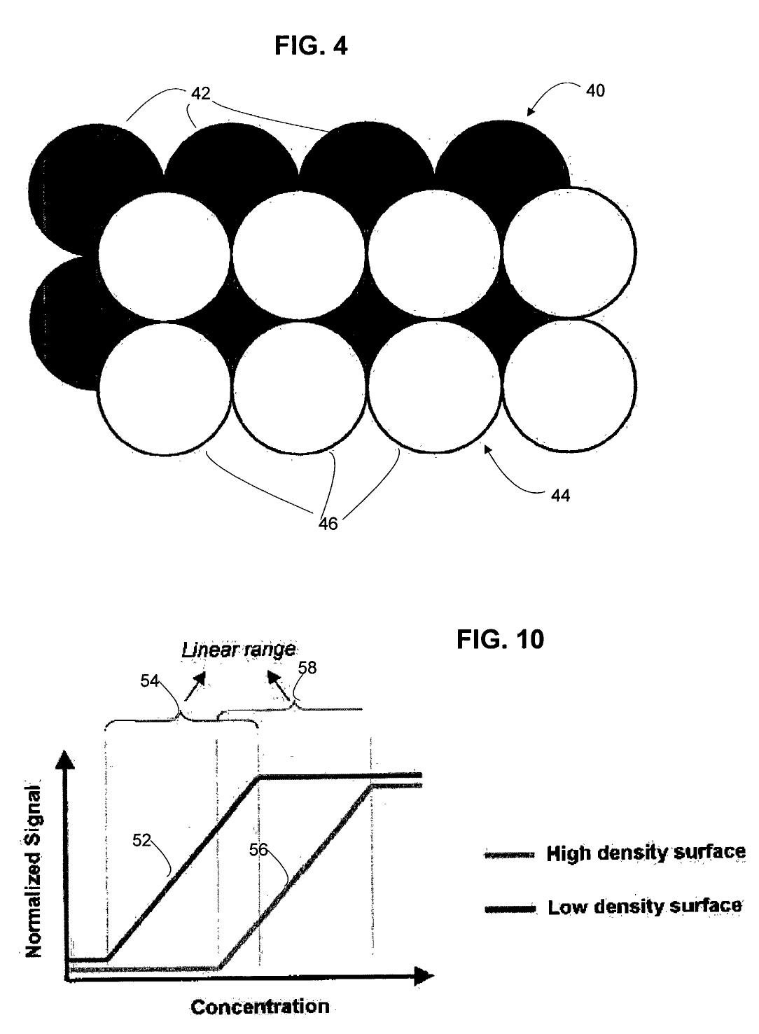

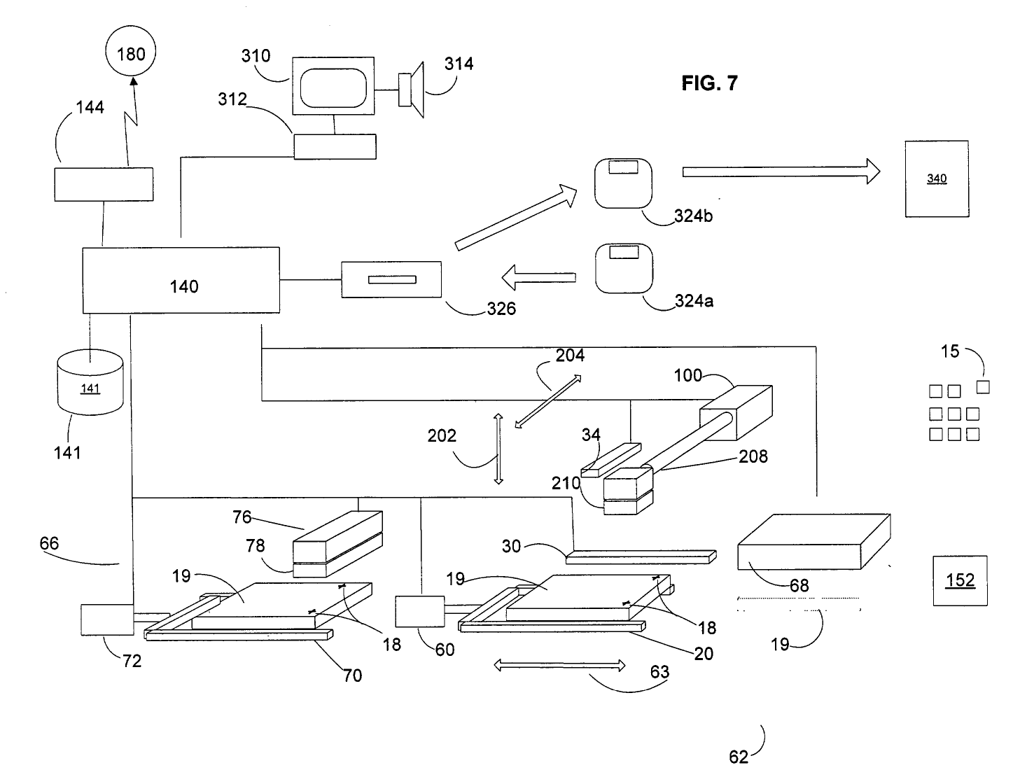

While in FIG. 1 the substrate surface has four regions (the regions occupied by arrays 12a, 12b, 12c, 12d) at which a linking agent is bound (two pairs of two regions at different densities) other configurations can be provided. For example, referring to FIG. 5 substrate 10 has two distinct regions each carrying an array 12e, 12f which have the same array layout and probe compositions but with different densities as already described. Similarly, substrate 10 in FIG. 6 has two arrays of different layout and probe compositions, with array 12g being repeated as arrays 12h to 12m in the regions coextensive with those arrays and which have successively higher feature probe densities and region linker agent densities, and successively lower capping agent density, going from array 12g to array 12m. Similarly, the different array 12n is repeated as arrays 12n to 12t in the regions coextensive with those arrays and which have successively higher feature probe densities and region linker agent densities, and successively lower capping agent density, going from array 12n to array 12t. Furthermore, regions of different linking agent density may carry parts of a single array (that is, the same array has regions of different probe feature density) with the features in one part being repeated at a different feature probe density in another part. For example, arrays 12a, 12c could be positioned immediately adjacent each other to form a single array. This can, for example, be accomplished where there is precise control over the boundaries of the different regions with different linker agent density, such as is possible with the apparatus and methods described below in connection with FIGS. 4 and 7.

To fabricate arrays of FIGS. 1-3, 5, 6 a surface modified substrate 10 may first be produced. Such a substrate will have the characteristics described above but with no features (thus, no probes) present in each region.

### Methods of Making Surface Modified Substrates and Arrays

One way of producing surface modified substrates as described above is to first modify front surface 11a of substrate 10 by depositing drops containing a linking agent (such as a linking agent and a suitable solvent) onto the surface, so that the linking agent will bind to the substrate surface. Additionally, drops may be deposited which include a capping agent onto the surface, which drops may be the same or different from those containing the linking agent. When the same drops are used those with a higher concentration of linking agent will generally contain a lower concentration of capping agent. In either event the deposited drops containing the linking agent or the capping agent are of such a size, and are sufficiently close together, so that together such drops cover a desired region over the surface. For example, at least 10, at least 100, at least 200, or at least 1000 drops deposited at different locations on the surface will together cover a region on surface 11a. By covering a “region” on the surface does not mean that the drops must simultaneously be in liquid form, but in fact some may have already dried. Instead they need only be of sufficient size and sufficiently close together such that the total region occupied by them (dry or not) is continuous. Drops size and spacing can be adjusted by known methods particularly when pulse jets are used to deposit the drops.

The linking agent and capping agent will bind to the different regions to produce a substrate surface having both linking agent and capping agent bound to the different regions with a lower density of capping agent at a region which has a higher density of linking agent. The different regions could be coextensive with the different arrays described above. For example, the there could be four regions each coextensive with arrays 12a, 12b, 12c, 12d in FIG. 1, with the regions for arrays 12a, 12b having a same region linking agent density and those regions for arrays 12c, 12d also having a same region linking agent density, but with regions for arrays 12a, 12b having a higher region linking agent density than the regions for arrays 12c, 12d. However, this need not necessarily be the case. For example, in the array assembly of FIG. 1 one half of surface 11a (that half carrying arrays 12a, 12b) could be produced with a higher linking agent density, while the other half (carrying arrays 12c, 12d) could be produced with a lower linking agent density. Similarly, in FIG. 6 rather than each region being coextensive with an individual array therein, each region could be formed as a stripe across the surface which will encompass two arrays of the same feature probe density (for example, one stripe will encompass arrays 12g, 12n, a next stripe will encompass arrays 12h, 12o and so on with linker agent density increasing in successive stripes moving from left to right in FIG. 6).

One procedure for producing a surface of desired linker agent and capping agent densities, is illustrated in FIG. 4. In FIG. 4 one or more heads such as head 210 (see below) makes one pass to deposit drops of a liquid (for example, solvent, linking agent, or additional agent). In FIG. 4 eight drops 42 have been deposited to form one continuous layer 40. To ensure no gaps are present the head makes another pass to deposit the same liquid in eight drops 46 to form another continuous layer 44. Thus drops 42 and 46 together cover a continuous area (the union of the areas represented by layers 40 and 44) with no gaps. In practice, the continuous area may be the entire area of a substrate surface, or at least 50%, 25%, 10% or 5% of such area. Regardless of the foregoing, the continuous area will normally cover at least the continuous area occupied by multiple adjacent features of an array 12 to be fabricated on the substrate or the entire continuous area occupied by an array 12, with different continuous areas for different arrays 12. Alternatively, the continuous area may be at least 0.1 cm², at least 0.2 cm², or at least 0.5 cm² or 1 cm².

One way to produce a surface modified substrate by a method of the present invention, drops which include a solvent may be fist deposited onto the substrate surface to cover a continuous area using the method as described in connection with FIG. 4. Drops which include the linking agent may then be deposited in the manner described in connection with FIG. 4 also to cover a same continuous area as the solvent deposited drops. These drops may include a capping agent or such capping agent may be in drops deposited later to cover a same continuous area as covered by the drops containing the linking agent. In either event, the linking agent binds to the surface so that probe or probe precursors subsequently deposited in further drops at aim feature locations for an array 12 will bind to the linking agent but not to the capping agent. However, further processing of a functional group on a linking agent may be necessary for such binding to occur.

As to a suitable capping agents this may particularly be any of the first silanes as set out in detail U.S. Pat. No. 6,444,268, while the linking agent may be any of the second silanes therein and the solvent may be as described in that patent also (for example, toluene). As already mentioned, that patent is incorporated herein by reference, including for example the details of the first and second silanes and solvents used therein. In one embodiment as described in the foregoing patent the first silane has the formula R¹—Si(R^{L}R^{x}R^{y}) and the second silane has the formula R²-(L)ₙ-Si(R^{L}R^{x}R^{y}) so that binding to the surface provides —Si—R¹ groups and —Si-(L)ₙ-R² groups thereon, wherein the R^{L}, moieties, which may be the same or different, are leaving groups, the R^{x} and R^{y} are independently lower alkyl or leaving groups, R¹ is a chemically inert moiety that upon binding to the substrate surface lowers the surface energy thereof, n is 0 or 1, L is a linking group, and R² is a functional group enabling covalent binding of a molecular moiety or a modifiable group that may be converted to such a functional group. Leaving groups in the foregoing may include halogen and alkoxy. Both the first and second silanes bind to the surface through reactive hydrophilic moieties thereon, which are selected from the group consisting of hydroxyl, carboxyl, thiol, amino, and combinations thereof. The foregoing terms and other emobidments of the first and second silanes are further defined in the foregoing patent. However, in this method, unlike in the foregoing patent, drops of the solvent (for example, toluene) may be dispensed onto the surface 11a so that together they cover a continuous area with no gaps, as described in connection with FIG. 4. This may be followed by depositing drops of the second silane to cover the same continuous area. The drops containing the second silane may also contain the first silane. The substrate 10 may then be physically and chemically processed as described in detail in U.S. Pat. No. 6,444,268. If the second silane was not present in the drops containing the first silane, then drops containing the second silane may be deposited onto the substrate surface to cover the same continuous area at this point, and allowed to react therewith for about 30 minutes. In either situation, the relative amounts of the first and second silanes can be adjusted to control surface energy as also described in detail in U.S. Pat. No. 6,444,268.

In one example, two printheads 210 may be used, the first to deposit a toluene/water solvent mixture, and a second to deposit the pure or diluted mixture of the first and second silanes. Due to the low surface tension of toluene, only a few passes of the printheads are needed to obtain complete coverage of a front surface 11a of substrate 10. In particular, drops of the solvent and silane mixture are each applied in a pattern similar to that shown in FIG. 4. A total volume of 5 ml will form a 200 μm thick continuous layer of toluene/water solution or silane solution on a 6 inch by 6 inch substrate, and at the concentrations described in the foregoing patent the silane containing layer will contain enough silane to completely react with all sites on the wetted substrate surface. The substrate is then placed in a holding chamber for about 20 minutes to allow the reaction (covalent binding of the first and second silanes with reactive hydroxyls on the substrate surface) to go to completion.

In a second example, three printheads 210 may be used. One is used for depositing drops of the toluene/water mixture, and another one each for pure or diluted first and second silanes. This example is the same as in the first example, except here drops containing the first and second silanes are separately deposited. In particular, after deposition of the toluene/water solvent containing drops, drops containing the second silane are then deposited to provide a continuous layer over the substrate front surface (again, about 5 ml can be used). The concentration of the second silane is adjusted to react with only a fraction of the total sites available for reaction on the front surface. After 20 minutes waiting time at room temperature, the substrate is rinsed in toluene and dried. At this point, the linking agent will be linked to a portion of the sites on the front surface. Surface functionalization is then completed by depositing drops of toluene/water then drops containing the first silane, with the first silane concentration adjusted in excess to react with 1000 times the total sites available on the front surface of the substrate. After application the substrate is placed in a holding chamber for about 20 minutes to allow the reaction (covalent binding of the first silane to the reactive hydroxyls on the surface) to go to completion. This method will help to inhibit any artifacts resulting from “beading” of the solution as the formation of the self-assembling monolayer of silane progresses in the presence of the first silane, and will also help inhibit formation of islands at the molecular level which might otherwise reduce the effective local concentration of hydroxyl terminated second silane (following boration and oxidation as described in U.S. Pat. No. 6,444,268).

Note that in both examples the atmosphere in the holding chamber is controlled to prevent excessive evaporation or surface contamination Also, if needed, the process of either example can be repeated to ensure adequate functionalization of the substrate surface with the second silane (as well as an appropriate concentration of the first silane to obtain the desired surface energy quality). The substrate resulting from either example may then be used to fabricate an array using drop deposition in an in situ or another process (for example, deposition of previously obtained probe moieties, such as polynucleotides or proteins). This may be done by depositing onto the continuous functionalized area on the substrate surface, drops containing the chemical probes or probe precursors at the multiple feature locations of the array to be fabricated, so that the probes or probe precursors bind to the linking agent at the feature locations. This step may be repeated at one or more features, particularly when the in situ method of fabricating biopolymers is used. Such methods and their chemistry are described in detail in the references cited in the “Background” section above, including for example U.S. Pat. No. 6,242,266, U.S. Pat. No. 6,232,072, U.S. Pat. No. 6,180,351, U.S. Pat. No. 6,171,797, U.S. Pat. No. 6,323,043, U.S. and U.S. patent application Ser. No. 09/302,898 filed Apr. 30, 1999 by Caren et al., and the references cited in them.

Use of the foregoing method provides a relatively uniform coating within a region easier than could be obtained by exposing the substrate to the silanes in a reaction chamber. Furthermore, when a reaction chamber is used in which the silanes are introduced as a volume of solution into the chamber to cover the substrate surface, flow characteristics within the chamber can result in variation of linker density over the surface. Such variations can lead to variations in probe density (versus the expected probe density) within a feature, across the array, and between arrays. This is particularly true where it may be desirable to inject the silanes into the solvent within the chamber, rather than as part of a solvent solution with which the chamber is filled, to reduce possible polymerization of the silanes. As substrates become larger, these problems with using a reaction chamber may become more severe. On the other hand, increases in substrate size in a method of the present invention will not have the same effect. Additionally, with a method of the present invention, unlike a reaction chamber, it is not necessary to functionalize an entire surface of the substrate. Instead, only those parts of a substrate surface on which arrays will be fabricated need to be functionalized. Other areas of the same substrate surface are then available for further reaction or use, such as for application of a silicon glue to form a gasket to retain a hybridization solution when mated with a cover, or for printing of bar codes 356 or fiducial marks 18. However, the present invention contemplates that other methods such as the, reaction chamber, or dipping tanks for the different solutions, could alternatively be used.

Arrays of the present invention of a type already described above in connection with FIGS. 1-3, 5, 6, can be produced by depositing drops containing the same concentration of different biopolymers or biopolymer precursors (for example, biomonomers such as nucleoside phosphoramidites) onto the feature locations for the different arrays. Such procedures are disclosed in detail in U.S. in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. As already mentioned, these references are incorporated herein by reference. Other drop deposition methods could also be used for array fabrication. Also, instead of drop deposition methods, photolithographic array fabrication methods may be used such as described in U.S. Pat. No. 5,599,695, U.S. Pat. No. 5,753,788, and U.S. Pat. No. 6,329,143. Interfeature areas need not be present particularly when the arrays are made by photolithographic methods as described in those patents.

One particular apparatus of the present invention for producing surface modified substrates and arrays according to a method of the present invention, is described in detail below. Additionally, an apparatus and methods for reading arrays according to the present invention are also described.

### Apparatus for Producing Surface Modified Substrates and Arrays

Referring now to FIG. 7, an apparatus of the present invention which can execute a method of the present invention, is illustrated. This apparatus is configured for use with a large substrate 19 which will later be cut into individual substrates 10 of any of the array assemblies 15. Substrate 19 will therefore also be referred to as having surfaces 11a and 11b. The apparatus shown essentially has two sections, a first section on which a surface 11a of the substrate 19 can be functionalized, and a second section in which the array is fabricated on the functionalized surface of the substrate 19. While these two sections are shown as part of one apparatus in FIG. 7, it will be appreciated that they can be entirely separate with the first section preparing many functionalized substrates 19 which are forwarded to the fabrication section for array fabrication, with their possibly being one or more first sections and one or more second sections remote from each other.

The first section of the apparatus of FIG. 7 includes a first substrate station 70 which can retain a mounted substrate 19, a third transporter 70, a head retainer 76, and a first drop deposition system in the form of a stationary pulse jet head 78 system. Pulse jet head system 78 can include two or three pulse jet heads which deliver drops of the solvent, linking agent, and additional agent, onto surface 11a of substrate 19 all as already described, so as to functionalize that surface. Drops are delivered from the stationary pulse jet head 78 while substrate 19 is advanced beneath it by transporter 70, all under control of a processor 140. A suitable holding chamber (not shown) may also be provided for the purposes already described during such functionalization. A mechanical means (such as a robot arm) may be provided to transfer a substrate 19 from substrate station 10 to the holding chamber, and to a second substrate station 20 when functionalization of the surface 11a is complete.

The second section of the apparatus of FIG. 7 includes substrate station 20 (sometimes referenced as a “substrate holder”) on which a substrate 19 can be mounted and retained. Pins or similar means (not shown) can be provided on substrate station 20 by which to approximately align substrate 19 to a nominal position thereon (with alignment marks 18 on substrate 19 being used for more refined alignment). Substrate station 20 can include a vacuum chuck connected to a suitable vacuum source (not shown) to retain a substrate 19 without exerting too much pressure thereon, since substrate 19 is often made of glass. A flood station 68 is provided which can expose the entire surface of substrate 19, when positioned at station 68 as illustrated in broken lines in FIG. 7, to a fluid typically used in the in situ process, and to which all features must be exposed during each cycle (for example, oxidizer, deprotection agent, and wash buffer). In the case of deposition of a previously obtained polynucleotide, flood station 68 need not be present.

A second drop deposition system is present in the form of a dispensing head 210 which is retained by a head retainer 208. As mentioned above though, the head system can include more than one head 210 retained by the same head retainer 208 so that such retained heads move in unison together. The transporter system includes a carriage 62 connected to a first transporter 60 controlled by processor 140 through line 66, and a second transporter 100 controlled by processor 140 through line 106. Transporter 60 and carriage 62 are used execute one axis positioning of station 20 (and hence mounted substrate 19) facing the dispensing head 210, by moving it in the direction of axis 63, while transporter 100 is used to provide adjustment of the position of head retainer 208 (and hence head 210) in a direction of axis 204 (and therefore move head 210 in the direction of travel 204a which is one direction on axis 204). In this manner, head 210 can be scanned line by line along parallel lines in a raster fashion, by scanning along a line over substrate 19 in the direction of axis 204 using transporter 100, while line to line transitioning movement of substrate 19 in a direction of axis 63 is provided by transporter 60. Transporter 60 can also move substrate holder 20 to position substrate 19 in flood station 68 (as illustrated by the substrate 19 shown in broken lines in FIG. 7). Head 210 may also optionally be moved in a vertical direction 202, by another suitable transporter (not shown) and its angle of rotation with respect to head 210 also adjusted. It will be appreciated that other scanning configurations could be used during array fabrication. It will also be appreciated that both transporters 60 and 100, or either one of them, with suitable construction, could be used to perform the foregoing scanning of head 210 with respect to substrate 19. Thus, when the present application recites “positioning”, “moving”, or similar, one element (such as head 210) in relation to another element (such as one of the stations 20 or substrate 19) it will be understood that any required moving can be accomplished by moving either element or a combination of both of them. The head 210, the transporter system, and processor 140 together act as the deposition system of the apparatus. An encoder 30 communicates with processor 140 to provide data on the exact location of substrate station 20 (and hence substrate 19 if positioned correctly on substrate station 20), while encoder 34 provides data on the exact location of holder 208 (and hence head 210 if positioned correctly on holder 208). Any suitable encoder, such as an optical encoder, may be used which provides data on linear position.

Processor 140 also has access through a communication module 144 to a communication channel 180 to communicate with a remote station. Communication channel 180 may, for example, be a Wide Area Network (“WAN”), telephone network, satellite network, or any other suitable communication channel.

Each of one or more heads 210 may be of a type similar to that used in an ink jet type of printer and may, for example, include five or more chambers (at least one for each of four nucleoside phosphoramidite monomers plus at least one for an activator solution) each communicating with a corresponding set of multiple drop dispensing orifices and multiple ejectors which are positioned in the chambers opposite respective orifices. Each ejector is in the form of an electrical resistor operating as a heating element under control of processor 140 (although piezoelectric elements could be used instead). Each orifice with its associated ejector and portion of the chamber, defines a corresponding pulse jet. It will be appreciated that head 210 could, for example, have more or less pulse jets as desired (for example, at least ten or at least one hundred pulse jets, with their nozzles organized in rows and columns). Application of a single electric pulse to an ejector will cause a droplet to be dispensed from a corresponding orifice. Certain elements of the head 210 can be adapted from parts of a commercially available thermal inkjet print head device available from Hewlett-Packard Co. as part no. HP51645A. A suitable head construction is described in U.S. Pat. No. 6,461,812, incorporated herein by reference. Alternatively, multiple heads could be used instead of a single head 210, each being similar in construction to head 210 and being movable in unison by the same transporter or being provided with respective transporters under control of processor 140 for independent movement. In this alternate configuration, each head may dispense a corresponding biomonomer (for example, one of four nucleoside phosphoramidites) or an activator solution.

Each head of head system 78 may also be of a type similar to that of each head 210, as already described. However, since each head will deliver only liquid drops of one type (solvent, or one of the two silanes) each head need only have one chamber to provide fluid to all the pulse jets of that head.

As is well known in the ink jet print art, the amount of fluid that is expelled in a single activation event of a pulse jet, can be controlled by changing one or more of a number of parameters, including the orifice diameter, the orifice length (thickness of the orifice member at the orifice), the size of the deposition chamber, and the size of the heating element, among others. The amount of fluid that is expelled during a single activation event is generally in the range about 0.1 to 1000 pL, usually about 0.5 to 500 pL and more usually about 1.0 to 250 pL. A typical velocity at which the fluid is expelled from the chamber is more than about 1 m/s, usually more than about 10 m/s, and may be as great as about 20 m/s or greater. As discussed above, when the orifice is in motion with respect to the substrate surface at the time an ejector is activated, the actual site of deposition of the material will not be the location that is at the moment of activation perpendicularly aligned with an orifice. However, the actual deposited location will be predictable for the given distances and velocities.

The apparatus further includes a display 310, speaker 314, and operator input device 312. Operator input device 312 may, for example, be a keyboard, mouse, or the like. Processor 140 has access to a memory 141, and controls print head system 78 and print head 210 (specifically, the activation of the ejectors therein), operation of the transporter system and the third transporter 72, and operation of display 310 and speaker 314. Memory 141 may be any suitable device in which processor 140 can store and retrieve data, such as magnetic, optical, or solid state storage devices (including magnetic or optical disks or tape or RAM, or any other suitable device, either fixed or portable). Processor 140 may include a general purpose digital microprocessor suitably programmed from a computer readable medium carrying necessary program code, to execute all of the steps required by the present invention, or any hardware or software combination which will perform those or equivalent steps. The programming can be provided remotely to processor 141 through communication channel 180, or previously saved in a computer program product such as memory 141 or some other portable or fixed computer readable storage medium using any of those devices mentioned below in connection with memory 141. For example, a magnetic or optical disk 324a may carry the programming, and can be read by disk writer/reader 326. A cutter 152 is provided to cut substrate 19 into individual array assemblies 15.

### Operation of the Apparatus

The operation of the apparatus of FIG. 7 will now be described. Reference numbers in parentheses refer to FIG. 10. In a first step of the sequence, a substrate surface will be produced (500) with surface regions of different linker agent density (and different capping agent density, as discussed above). It will be assumed that a substrate 19 is already mounted on substrate station 70. In this case the substrate is functionalized using the toluene/water solvent, and first and second silanes, in accordance with the method already described. To accomplish this transporter system 72 advances the mounted substrate 19 beneath head system 78 while drops of solvent are deposited to provide the continuous layer of solvent with no gaps, as already described. This process can be repeated but with drops containing the second silane then being deposited. The first silane may be included in the drops with the second silane or the foregoing can be repeated in the case where the first silane is delivered as drops of a separate solution. The substrate may be transferred to the holding chamber between the second and first silane (if separate) and following application of both, using a robot arm or manually by an operator, in accordance with the method as already described.

At this point preparation of the functionalized surface 11a is complete.

The substrate 19 with the functionalized surface 11a may then be transferred to the substrate station 20 either manually or by the robot arm, as which station one or more arrays will be fabricated (520) on the substrate surface 11a with features in one region repeated in another different region at a different feature probe density. In this sequence it will be assumed that processor 140 is programmed with the necessary layout information to fabricate target arrays 12. Using information such as the foregoing target layout and the number and location of drop dispensers in head 210, processor 140 can then determine a reagent drop deposition pattern. Alternatively, such a pattern could have been determined by another processor (such as a remote processor) and communicated to memory 141 through communication channel 180 or by forwarding a portable storage medium carrying such pattern data for reading by reader/writer 326. Processor 140 controls fabrication, in accordance with the deposition pattern, to generate the one or more arrays 12 on each section of substrate 19 which will later be cut into each substrate 10, by depositing for each target feature during each cycle, a reagent drop set as previously described. This is repeated at each of the different desired regions on the surface 11a for a substrate 10 (for example, the regions at each of the regions at which arrays 12a, 12b, 12c, 12d will be formed) so that the probe or probe precursors bind to the different regions through the linker agent. The foregoing sequence is repeated for each cycle of the in situ fabrication process. Drops are deposited from the head while moving along each line of the raster during scanning. No drops are dispensed for features or otherwise during line transitioning. Processor 140 also sends substrate 19 to flood station 68 for cycle intervening or final steps as required, all in accordance with the conventional in situ polynucleotide array fabrication process described above.

As a result of the above, multiple array assemblies are formed on each section which will be cut to form a substrate 10, so as to form the array thereon with features of different probe composition in a region which features are repeated in another region but with a different probe density.

The substrate 19 may then be sent to a cutter 152 wherein sections of substrate 19 are separated into substrates 10 carrying one ore more arrays 12, to provide multiple array assemblies 15. One or more array assemblies 15 may then be forwarded to one or more remote users. Processor 140 also causes deposition of drops from all multi-dispenser drop groups to be deposited at separate test locations, such as at a test pattern 250 which may be separate from arrays 12 as already described above. The foregoing array fabrication sequence can be repeated at the fabrication station as desired for multiple substrates 19 in turn.

During array fabrication errors can be monitored and used in any of the manners described in U.S. patent application “Polynucleotide Array Fabrication” by Caren et al., Ser. No. 09/302898 filed Apr. 30, 1999, and U.S. Pat. No. 6,232,072. Also, the one or more identifiers in the form of bar codes 356 can be attached or printed onto sections of substrate 19 defining the substrates 10 before entering, or after leaving, first fabrication station 70, or after leaving the second fabrication station 20. If bar codes 356 are present before entering first fabrication station 70 they can include an indication of the location of different regions on a substrate to which probes or probe precursors will bind with different density. They can then be read by a bar code reader (not shown) in the first fabrication station, and received by processor 140 to then control the drop deposition system to form the one or more arrays with features of different probe composition in one of the regions which are repeated in another of the regions at a different feature probe density. If bar codes 356 are present before substrate 190 enters second substrate station 20, they can be read by a bar code reader (not shown) in the second substrate station and used by processor 140 to determine the different regions at which features are to be repeated with a same probe composition which processor 140 will then form in second substrate station 20. Any of the foregoing types of information on the different regions can be contained within the bar codes 356 (or other identifiers) or in a file previously linked to them. Regardless of the foregoing, at any point in the operation of the apparatus of FIG. 7, processor 140 will associate (540) each array with an identifier such as a bar code 356, which identifier carries an indication of the different feature probe densities of the same probe composition or is linked to a file carrying such information. The file and linkage can be stored by processor 140 and saved into memory 141 or can be written onto a portable storage medium 324b which is then placed in the same package 340 as the corresponding array assembly 15 for shipping to a remote customer. The actual indication can take many forms. For example, one or more of the bar codes 356 associated with the arrays on the same substrate 10, may specify that one array 12a is the same as another array such as array 12c, but that one the region carrying one has a feature probe density which is a proportion of the region at the other (for example, the feature probe density of array 12c is 20% that of array 12a). Alternatively, absolute feature probe density numbers may be provided for each array in its associated bar code 356.

Optionally other characteristics of the fabricated arrays can be included in the code 356 applied to the array substrate or a housing, or a file linkable to such code, in a manner as described in the foregoing patent application and U.S. Pat. No. 6,180,351. As mentioned above, these references are incorporated herein by reference.

### Array Reading Apparatus

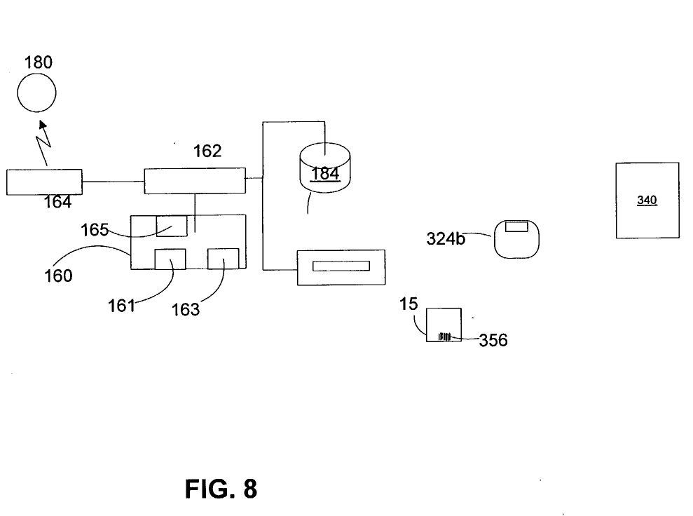

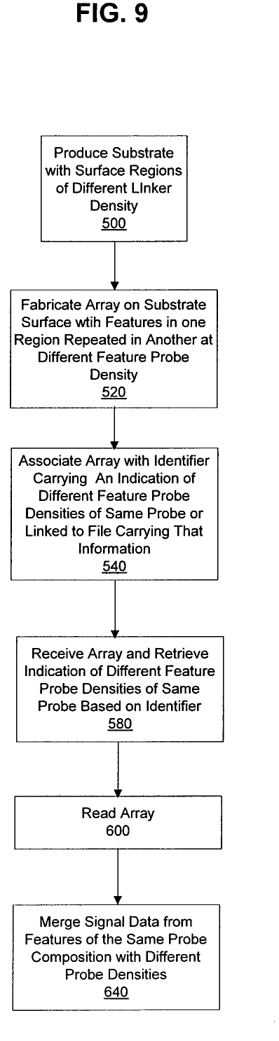

FIG. 8 illustrates an array reader at a single “user station”, which is likely to be (but not necessarily) remote from the fabrication station of FIG. 7 (usually the user station is at the location of the customer which ordered the received array 12). The user station includes a processor 162, a memory 184, a scanner 160 which can read an array, data writer/reader 186 (which may be capable of writing/reading to the same type of media as writer/reader 326), and a communication module 164 which also has access to communication channel 180. Processor 162 is programmed to perform all the functions required of it. Scanner 160 may include a holder 161 which receives and holds an array assembly in the form of an array unit 18 or in the form of web 10 carrying arrays 12, as well as a source of illumination (such as a laser) and one or more light sensors 165 to read fluorescent light signals from respective features on the array as signal data which is obtained by processor 162 from the light sensor. Scanner 160 also includes a reader 163 to read a bar code 356 appearing on array assembly 15. Processor 162 is also capable of identifying signal data from read features 16 of a same probe composition with different feature probe densities, based on the read indication from a read bar code 163, and merging signal data from such in a manner further described below.

Communication module 164 may be any type of suitable communication module, such as those described in connection with communication module 144. Memory 184 can be any type of memory such as those used for memory 141. Scanner 160 can be any suitable apparatus for reading an array, such as one which can read the location and intensity of fluorescence at each feature of an array following exposure to a fluorescently labeled sample. For example, such a scanner may be similar to the DNA MICROARRAY SCANNER available from Agilent Technologies, Inc. Palo Alto, Calif. Other suitable apparatus and methods are described in U.S. patent application Ser. No. 09/846125 “Reading Multi-Featured Arrays” by Dorsel et al.; and U.S. Pat. No. 6,406,849. The scanning components of scanner 160, holder 161, and reader 163 may all be contained within the same housing of a single same apparatus.

It will be understood that there may be multiple user stations such as shown in FIG. 8, each remote from the fabrication station and each other, in which case the fabrication station acts as a central fabrication station (that is, a fabrication station which services more than one remote user station at the same or different times). One or more such user stations may be in communication with the fabrication station at any given time. It will also be appreciated that processors 140 and 162 can be programmed from any computer readable medium carrying a suitable computer program. For example, such a medium can be any memory device such as those described in connection with memory 141, and may be read locally (such as by reader/writer 320 in the case of processor 140 or writer/reader 186 in the case of processor 162) or from a remote location through communication channel 180.

### Method of Using and Reading Arrays

At the user station of FIG. 8, the package 340 is then received (580) from the remote fabrication station and opened manually to retrieve an array assembly 15 and portable storage medium 324b (if any was present in package 340). A contiguous layer of a sample, for example a test sample, is exposed to the one or more arrays 12 on the received array assembly 15 in a known manner under known conditions. Apparatus and procedures for hybridization are described, for example, in U.S. Pat. No. 6,258,593 and U.S. Pat. No. 6,399,394. In one configuration, a separate drop of each target containing solution may be placed on each circular array in the embodiment of FIG. 6. Following hybridization and washing in a known manner, the array unit 18 is then inserted into holder 161 in scanner 160 and read (600) by it to obtain read results (such as signal data representing the fluorescence pattern on the array 12). The reader 163 in scanner 160 also reads the identifier 356 present on the array assembly 15 in association with the corresponding array 12, while the array unit 18 is still positioned in retained in holder 161 or beforehand. Using identifier 356, processor 162 may then retrieve (580) the characteristic data for one or more arrays 12 from portable storage medium 324b or from the database of such information in memory 141 by communicating the map identifier to that database through communication module 164 and communication channel 180 and receiving the corresponding identity map in response. Such characteristic data may include the indication of features of different probe densities of a same probe composition as discussed above.

The resulting retrieved characteristic data for an array may be used to either control reading of the array or to process information obtained from reading the array. For example, the customer may decide (through providing suitable instructions to processor 162) that a particular feature need not be read or the data from reading that feature may be discarded, since the polynucleotide sequence at that feature is not likely to produce any reliable data under the conditions of a particular sample hybridization. However, processor 162 uses the retrieved indication of features of different probe densities of a same probe composition, to identify signal data from features of a same probe composition with different probe densities. With such signal data identified, processor 162 can then merge (640) signal data from features identified to be of the same probe composition but with different probe densities. This merging can take place according to any suitable routine which may be preprogrammed into processor 162 by a user, or retrieved from portable storage medium 324b or remotely from memory 141 based on a read bar code 356 (or other identifier) for the one or more arrays being read. In one routine, signal data from a feature of higher probe density which is greater than some predetermined signal (for example, 90% or 95% of the maximum signal which can be read from such a feature by the reader), may be rejected in favor of the signal data from a feature of a same probe composition of lower feature probe density provided it is greater than some predetermined signal (for example at least 5% or 10% of the maximum signal which can be read from such a feature by the reader). If signals from multiple features of a same probe composition are all within predetermined minimum and maximum values, then some statistical representation (for example, an average signal) can be calculated from all of such signals.

Results from the array reading can be further processed results, such as obtained by rejecting a reading for a feature which is below a predetermined threshold and/or forming conclusions based on the pattern read from the array (such as whether or not a particular target sequence may have been present in the sample). The results of the reading (processed or not) can be forwarded (such as by communication) to be received at a remote location for further evaluation and/or processing, or use, using communication channel 180 or reader/writer 186 and medium 190. This data may be transmitted by others as required to reach the remote location, or re-transmitted to elsewhere as desired.

The present invention can provide one or more arrays with good dynamic range. Without limiting the present invention it is thought this occurs as explained in connection with FIG. 10. In particular a feature (such as feature 16c in FIG. 3) of lower feature probe density may have a Normalized Signal versus the Concentration of the target in a sample which that feature detects, illustrated by plot 52. The sloped region represents the dynamic range 54 of such a feature. On the other hand, a feature of the same probe composition but of higher feature probe density (such as feature 16a in FIG. 3) may have a similar plot 56 with a dynamic range 58. Providing an array with both features though allows for a total dynamic range over the union of the ranges 54 and 58.

In a variation of the embodiments above, it is possible that each array assembly 15 may be contained with a suitable housing. Such a housing may include a closed chamber accessible through one or more ports normally closed by septa, which carries the substrate 10. In this case, the identifier for all arrays on a substrate 10 can be associated with them by being applied to the housing. It will also be appreciated that arrays may be read by any other method or apparatus than that described above, with other reading methods including other optical techniques (for example, detecting chemiluminescent or electroluminescent labels) or electrical techniques (where each feature is provided with an electrode to detect hybridization at that feature in a manner disclosed in U.S. Pat. No. 6,251,685, U.S. Pat. No. 6,221,583 and elsewhere). As to retrieving signal data from features (“feature extraction”) in which features and their corresponding signals are identified in an image of a read array, this can be performed using procedures such as described in U.S. patent application Ser. Nos. 09/589046, 09/659415 and 10/086839, all under the title “Method And System For Extracting Data From Surface Array Deposited Features”.

The substrate surface onto which the polynucleotide compositions or other moieties is deposited may be porous or non-porous, smooth or substantially planar, or have irregularities, such as depressions or elevations. The substrate may be of one material or of multi-layer construction. Also, instead of drop deposition methods for fabricating an array on the functionalized substrate, photolithographic array fabrication methods may be used such as described in U.S. Pat. No. 5,599,695, U.S. Pat. No. 5,753,788, and U.S. Pat. No. 6,329,143. Where a pattern of arrays is desired, any of a variety of geometries may be constructed other than the organized rows and columns of arrays 12 of FIG. 1. For example, arrays 12 can be arranged in a series of curvilinear rows across the substrate surface (for example, a series of concentric circles or semi-circles of spots), and the like. Similarly, the pattern of features 16 may be varied from the organized rows and columns of features in FIG. 2 to include, for example, a series of curvilinear rows across the substrate surface(for example, a series of concentric circles or semi-circles of spots), and the like. While various configurations of the features can be used, the user should be provided with some means (for example, through the array identifier) of being able to ascertain at least some characteristics of the features (for example, any one or more of feature composition, location, size, performance characteristics in terms of significance in variations of binding patterns with different samples, or the like). The configuration of the array may be selected according to manufacturing, handling, and use considerations. The present methods and apparatus may be used to fabricate and use arrays of other biopolymers, polymers, or other moieties on surfaces in a manner analogous to those described above. Accordingly, reference to polymers, biopolymers, or polynucleotides or the like, can often be replaced with reference to “chemical moieties”.

Various further modifications to the particular embodiments described above are, of course, possible. Accordingly, the present invention is not limited to the particular embodiments described in detail above.

What is claimed is:

1. A method of fabricating one or more arrays of multiple chemical probes bound to a surface of a substrate at different features of the array, comprising:
(a) contacting each of at least ten different probes or probe precursors with different locations on the substrate surface, so that each of the probes or probe precursors binds to the different locations through the linker agent; and
(b) repeating (a) as needed;
so as to form the one or more arrays with at least ten features of different probe composition which are repeated with a different probe density.

2. A method according to claim 1 wherein the probes are polynucleotides.

3. A method according to claim 1 wherein fluids containing the probes or probe precursors are contacted with the substrate surface, and wherein a same concentration of a same probe or probe precursor contacts the locations of repeated features of different probe density.

4. A method according to claim 1 wherein the one or more arrays are formed on different regions each of which has multiple features with a same probe density within a region and different probe densities in different regions.

5. A method according to claim 4 wherein the different regions each has at least one hundred features of a same probe density.

6. A method according to claim 5 wherein there are at least three different regions.

7. A method according to claim 1 wherein the probes or probe precursors are contacted with the surface locations by depositing drops comprising the probes or probe precursors at the multiple feature locations.

8. A method according to claim 1 additionally comprising associating the one or more arrays with an indication of the different probe densities of a same probe.

9. A method according to claim 8 wherein the one or more arrays are associated with the indication by providing one or more identifiers on the substrate which carry the indication or providing the one or more identifiers and linking them to a file which carries the indication.

10. A method of fabricating one or more arrays of multiple chemical probes bound to a surface of a substrate at different features of the array, comprising:
(a) contacting each of different probes or probe precursors with different regions of the substrate surface which bind the same probes or probe precursors at different densities, so that the probe or probe precursors bind to the different regions; and
(b) repeating (a) as needed;
so as to form the one or more arrays with features of different probe composition in a region which are repeated in another region at a different probe density.

11. A method according to claim 10 wherein probe precursors repeatedly contact the different feature locations.

12. A method of fabricating one or more arrays of multiple chemical probes bound to a surface of a substrate at different features of the array, comprising:
(a) contacting each of different probes or probe precursors with different regions of the substrate surface at which a linking agent is bound at different densities, so that the probe or probe precursors bind to the different regions through the linker agent; and
(b) repeating (a) as needed;
so as to form the one or more arrays with features of different probe composition in a region which are repeated in another region but with a different probe density.

13. A method according to claim 12 wherein multiple features of higher probe density are formed on a region of higher linking agent density and multiple features of lower probe density are formed on a region of lower linking agent density.

14. A method according to claim 12 wherein the different regions of different linking agent density also have a capping agent with a lower density of capping agent at a region which has a higher density of linker agent.

15. A method according to claim 12 wherein each region of different linking agent density has an area of at least 10 mm².

16. A method according to claim 13 wherein at least one hundred features of different probe composition are formed on region of higher linking agent density which features are repeated at a lower probe density on a region of lower linking agent density.

17. A method according to claim 12 wherein the probes or probe precursors are contacted with the surface by depositing drops comprising the probes or probe precursors at the multiple feature locations.

18. A method according to claim 13 wherein:
the probes or probe precursors are contacted with the surface by depositing drops comprising the probes or probe precursors at the multiple feature locations; and
the different regions have different contact angles with drops of the same composition comprising the probe or probe precursors;
drops of a larger volume are deposited on one of the regions having a higher contact angle than are deposited at another one of the regions having a lower contact angle.

19. A method according to claim 12 wherein the substrate surface has at least three regions at which a linking agent is bound at different densities, and wherein features of higher probe density are formed on each of the regions of higher linking agent density and features of lower probe density are formed on each of the region of lower linking agent density.

20. One or more arrays of multiple chemical probes bound to a surface of a substrate at different features of the array, comprising at least ten features of different probe composition which are repeated at a different probe density.

21. One or more arrays according to claim 20 wherein the probes are polynucleotides.

22. One or more arrays according to claim 20 wherein different regions of the surface each has multiple features with a same probe density within a region and the different regions have different probe densities.

23. One or more arrays according to claim 22 wherein the different regions each has at least one hundred features of a same probe density.

24. One or more arrays according to claim 23 wherein there are at least three different regions.

25. An array assembly comprising one or more arrays according to claim 20 and an associated indication of features having different probe densities of a same probe composition.

26. An array assembly according to claim 25 wherein the associated indication comprises one or more identifiers on the substrate which carry the indication or one or more identifiers on the substrate which are linked to a file which carries the indication.

27. One or more arrays of multiple chemical probes bound to a surface of a substrate at different features of the array, comprising:
a linker agent bound to different regions of the substrate surface at different densities;
wherein the probes are bound to the different regions through the linker agent with features of different probe composition in a region which are repeated in another region but with a different probe density.

28. One or more arrays according to claim 27 wherein multiple features of higher probe density are formed on a region of higher linking agent density and multiple features of lower probe density are formed on a region of lower linking agent density.

29. One or more arrays according to claim 27 wherein the different regions of different linking agent density also have a capping agent, with a lower density of capping agent at a region which has a higher density of linker agent.

30. One or more arrays according to claim 27 wherein each region of different linking agent density has an area of at least 10 mm².

31. One or more arrays according to claim 28 wherein at least one hundred features of different probe composition are present on region of higher linking agent density which features are repeated at a lower probe density on a region of lower linking agent density.

32. One or more arrays according to claim 28 wherein the different regions have different contact angles with drops of a same composition.

33. One or more arrays according to claim 27 wherein the substrate surface has at least three regions at which a linking agent is bound at different densities, and wherein features of higher probe density are formed on each of the regions of higher linking agent density and features of lower probe density are formed on each of the region of lower linking agent density.

34. A method comprising:
reading one or more arrays according to claim 20 each of which has been exposed to a sample, to obtain signal data from the features; and
merging signal data from features of the same probe composition with different probe densities.

35. A method comprising:
reading one or more arrays of an array assembly according to claim 25 each of which has been exposed to a sample, to obtain signal data from the features;
identifying signal data from features of a same probe composition with different probe densities, based on the indication; and
merging signal data from features identified to be of the same probe composition with different probe densities.

36. A method comprising:
reading one or more chemical arrays of an array assembly according to claim 25 each of which has been exposed to a sample, to obtain signal data from the features;
reading the one or more identifiers and retrieving the indication of features of different probe densities of a same probe composition;
identifying signal data from features of a same probe composition with different probe densities, based on the indication; and
merging signal data from features identified to be of the same probe composition with different probe densities.

37. A method of producing a surface modified substrate comprising contacting different regions of the substrate surface to a linking agent under different conditions so that the linking agent binds to the different regions at different densities.

38. A method according to claim 37 wherein the different regions of the substrate are contacted with fluids having different concentrations of the linking agent.

39. A method according to claim 37 wherein each region is at least 10 mm² in area.

40. A method according to claim 38 additionally comprising contacting the different regions of the substrate with a capping agent so that the capping agent will also bind to the different regions to produce a substrate surface having both linking agent and capping agent bound to the different regions with a lower density of capping agent at a region which has a higher density of linking agent.

41. A method according to claim 40 wherein the capping agent is in the same fluids as the linking agent which contacts the substrate surface, a fluid with a higher concentration of linking agent containing a lower concentration of capping agent.

42. A method according to claim 38 wherein the contacting of fluids with different concentrations of linking agent with the different regions of the substrate comprises depositing drops of the different fluids at the different regions so that the drops for each region together continuously cover that region.

43. A method according to claim 40 wherein the capping agent is in a different fluid from the linking agent fluids and wherein the contact of the different linking agent fluids and capping agent fluid with the different regions of the substrate each comprises depositing drops of the fluid at the different regions so that the drops for each region together continuously cover that region.

44. An apparatus for fabricating one or more arrays each having multiple chemical probes bound to a surface at different features of the array, comprising:
(a) a substrate holder on which the substrate can be mounted;
(b) a drop deposition system to deposit drops of the probes or probe precursors; and
(c) a processor which controls the drop deposition systems so as to:
(i) contact each of at least ten different probes or probe precursors with different locations on the substrate surface; and
(ii) repeat (i) as needed;
so as to form the one or more arrays with at least ten features of different probe composition which are repeated with a different probe density.

45. An apparatus according to claim 44 wherein the processor receives an indication of the location of different regions on a substrate to which probes or probe precursors will bind with different density, and controls the drop deposition system to form the array with features of different probe composition in one of the regions which are repeated in another of the regions at a different probe density.

46. A computer program product for use with an apparatus for fabricating one or more arrays having multiple chemical probes bound to a surface at different features of the array, the program product comprising a computer readable storage medium having a computer program stored thereon which controls the apparatus to perform the method of claim 1.

47. A computer program product for use with an apparatus for fabricating one or more arrays having multiple chemical probes bound to a surface at different features of the array, the program product comprising a computer readable storage medium having a computer program stored thereon which controls the apparatus to perform the method of claim 10.

48. An apparatus for reading one or more chemical arrays of claim 20 each of which has been exposed to a sample, the apparatus comprising:
an array reader which reads the one or more arrays to obtain signal data from the array features; and
a processor which merges signal data from features of the same probe composition with different probe densities.

49. An apparatus for reading a chemical array of an array assembly according to claim 25 wherein each of the arrays has been exposed to a sample, the apparatus comprising:
an array reader which reads the one or more arrays to obtain signal data from the array features; and
a processor which:
obtains signal data from the features;
identifies signal data from features of a same probe composition with different probe densities, based on the indication; and
merges signal data from features identified to be of the same probe composition with different probe densities.
